(19) United States
(12) Patent Application Publication
(10) Pub. No.: US 2010/0159611 A1
(43) Pub. Date: Jun. 24, 2010

(54) HYDRATION/DEHYDRATION SENSOR

(76) Inventors: Xuedong Song, Alpharetta, GA (US); James M. Takeuchi, Roswell, GA (US)

Correspondence Address: KIMBERLY-CLARK WORLDWIDE, INC.;Tara Pohlkotte, 2300 Winchester Rd., NEENAH, WI, 54956, US

(21) Appl. No.: 12/338,673

(22) Filed: Dec. 18, 2008

(51) Int. Cl. G01N 33/84; G01N 31/22

(52) U.S. Cl. 436/163; 422/56

(57) ABSTRACT

A fluidic assay device or test format that can regulate or control the sample flow rate and modulate the manifestation of test results to reduce or eliminate errors is described. The assay device has a substrate with a flow-rate control zone that regulates the amount of time needed for development and appearance of a visual signal in the observation-feedback zone until the color transition in the detection zone reaches color stability. The present invention also describes absorbent articles incorporating such an assay device and methods of monitoring dehydration or testing ion strength of a urine sample using such a test format.

### FIELD OF INVENTION

The present invention relates to a sensor and absorbent products containing the sensor. In particular, the invention pertains to a sensor that can monitor a user's hydration status.

### BACKGROUND

Dehydration is the depletion of fluids and associated electrolytes from the body. Normally, a person's daily, total fluid amount is regulated to be within about ±0.02% of body weight, and water in the bode may comprise approximately 63% of the entire body mass. A balance of bodily fluids is achieved and maintained by matching the input and excretion of liquid from the body, and an imbalance in fluids can be linked to either dehydration or hypohydration. Dehydration can be of particular concern for either the infirm, elderly, or infants, and can have serious consequences to a dehydrated person if not cared for properly. Loss of body fluids in amounts of less than about 2-5% body mass have been associated with reduced heat dissipation, loss of cardiovascular function, and decreased physical stamina.

Specific gravity of an individual's urine is a routinely measured means of evaluating the relative hydration status of the individual. Determination of urine volume and electrolyte concentrations can aid in monitoring whether the individual's body fluid amounts are in balance. Urine specific gravity (USG) refers to the ratio of the density of urine to the density of water. USG is affected mainly by the solids and ions in urine. USG correlates proportionally with the solid concentration and ion concentration of urine. USG normally ranges from 1.002 to 1.030. It is accepted that USG<1.020 is considered to be well hydrated, USG between 1.020 and 1.025 is considered to be semi-dehydrated and USG>1.025 is considered to be severely dehydrated. USG can be measured by an instrument such as either a urinometer or urine test dipsticks or strips. Modern dipsticks are commonly based on lateral flow assay technology. Three major methods, namely refractometry, hydrometry and reagent strips, are commonly used for USG measurements. Although refractometry and hydrometry are very accurate, they require special instruments and trained persons to operate.

Over the years, various manufacturers have attempted different methods to improve the performance of the dipsticks for specific gravity, such as different formulations to increase sensitivity and specificity. Problems, however, persist for all the commercially available dipsticks. A major problem is that the user has to read a change in color within a few brief minutes after dipping in the sample because the color development is not stable under test conditions. The signals that one may observe outside of the time window are often inaccurate, hence normally invalid. For some analyte tests, such as ion concentration in urine (i.e., specific gravity for dehydration), a certain time period is needed before a signal is fully developed and a valid reading can be achieved. This situation may not be a problem for a test that a user can constantly monitor; however, it becomes a problem when constant monitoring of the test is not feasible and sample introduction time is uncertain. For instance, it is difficult, if not impossible, to predict accurately when a baby or incontinent adult will urinate to provide a sample for an assay device in a diaper or other personal care product. Therefore, the assay device requires a validation mechanism to make sure that a reading is within the valid reading time window.

In recent years, reagent strips have become more popular, particularly in the over-the-counter and point-of-care markets, mainly due to their low cost and ease of use. In general, conventional reagent strips change color in response to the ionic strength of a urine sample. The ionic strength of urine is a measure of the amount of ions present in the urine. The USG is proportional to the ionic strength of the urine. Therefore, by assaying the ionic strength of the test sample, the USG can be determined indirectly and semi-quantitatively by correlating the ionic strength of the urine to the USG.

Conventional reagent strips are usually made in such a way that all the relevant reagents are diffusively immobilized together on a small porous zone on the strip. A sample of urine is then applied to the zone or the entire strip is dipped in the urine sample and then pulled out quickly to allow color to develop. Examples of such conventional reagent strips are described in U.S. Pat. No. 4,318,709 to Falb et al. and U.S. Pat. No. 4,376,827 to Stiso et al.

U.S. Pat. No. 4,318,709 to Falb et al. and U.S. Pat. No. 4,376,827 to Stiso et al., both of which are incorporated by reference herein, describe the polyelectrolyte-dye ion exchange chemistry utilized in conventional test strips for measuring USG. In such conventional test strips, ions present in urine induce an ion-exchange with a polyelectrolyte, thereby introducing hydrogen ions into the urine. The change in hydrogen ion concentration is detected by a pH indicator.

However, conventional reagent strips for USG measurement suffer from major shortcomings, particularly for over-the-counter and point-of-care markets. For instance, conventional reagent strips have a limited reading window because the signal produced by such strips begins to change only a short period of time after sample application. Signal change can be caused by reagent leaching (the result of diffusively immobilized reagents) and sample evaporation. Unless the strips are analyzed shortly after application of the sample, the signal change can lead to erroneous test results. Furthermore, because the reagents in conventional strips are typically water soluble, the strips must also be pulled out quickly from the urine sample to prevent the reagents from leaching into the sample. In addition, conventional reagent strips are often designed for only a single urine sample application. Multiple urine insults can lead to erroneous test results making such strips unsuitable for applications in absorbent articles where multiple urine insults cannot be controlled. Finally, conventional reagent strips do not provide a way for a user to know if the test has been performed correctly or if enough sample has been applied.

Thus, an unsatisfied need exists for an assay device that can provide such assurance to caregivers in a cost effective way.

### SUMMARY OF THE INVENTION

The present invention pertains to a fluidic assay device or sensor that can regulate or control the sample flow rate and modulate the manifestation of test results to reduce or eliminate errors. The assay device has a first substrate with a porous matrix adapted for conducting lateral flow. The substrate has a sample contact zone, a detection zone, an observation-feedback zone, and a flow-rate control zone situated between the detection zone and feedback zone. Each of the respective zones is in fluidic communication with each other either directly or indirectly. The flow-rate control zone contains a separate, discrete substrate, such as a membrane or film, which can have a different porosity gradient or have a variety of flow-path features or micro-channels that help to regulate the progress of a sample volume from one section of the substrate to another. A supporting member secures each of the zones together in an integrate device.

The detection zone can be part of a buffer pad situated between the sample contact zone and the flow-rate control zone, or alternatively the observation-feedback zone, which is part of a wicking pad. The wicking pad may further include a sample observation-control zone that changes color upon contact with a urine sample, regardless of specific gravity of the urine. The flow-rate control zone regulates the flow rate from the buffer pad to the wicking pad. In some embodiments, the flow-rate control zone can be part of the same substrate as the wicking pad; but in other embodiments, the flow-rate control zone is at least part of a second substrate that separates from the first substrate. The flow-rate control zone has a porous membrane that bridges a gap between the buffer pad and the wicking pad. A variety of flow-rate control devices or mechanism may be arranged between the detection zone and the sample observation-feedback zone, with regions that overlap with the flow-rate control zone, to modulate or regulate the lateral flow of urine or other fluids as the fluid progresses from the deposition zone of the first or inner face to the detection zone of the second or outer face. The flow-rate control zone regulates the amount of time needed for development and appearance of a visual signal in the observation-feedback zone until the color transition in the detection zone reaches color stability. The mechanisms may take the form, for example, of micro-channels arranged in predetermined patterns and/or one or a number of differentiated substrate densities. These mechanisms may be oriented either parallel or orthogonal to the flow path of fluid. The flow-rate control zone regulates a predetermined time before the development of a visual signal in the observation-feedback zone so that color transition in the detection zone reaches color stability.

In another aspect, the present invention also describes a method of monitoring dehydration, the method comprises: providing a lateral flow strip with a porous matrix in fluid communication with a buffer pad, wicking pad, and a flow-rate control zone situated between said buffer pad and wicking pad; introducing a test sample to a sample zone on said buffer pad, allowing said sample to seep through a detection zone to said flow-rate control zone before developing a visual signal in an observation-feedback zone; controlling the flow rate by means of manipulating porosity, density, or ion affinity gradient in a matrix forming at least part of said flow-rate control zone.

Alternatively, a method for testing ion strength of a urine sample is provided. The method involves: introducing a urine sample to a sample zone, passing said urine through a buffer pad in a detection zone, causing a color change in a pH indicator in said detection zone, passing said urine through a flow-rate control zone to regulate the time needed for appearance of a visual signal in a control-feedback zone of said wicking pad until a color transition in said detection zone attains color stability

In another aspect, the present invention also relates to an absorbent article incorporating a lateral fluidic assay device as described above, for monitoring hydration or dehydration, and comprising: a first substrate with a porous matrix adapted for conducting lateral flow, the substrate having a sample contact zone, a detection zone, an observation-feedback zone, and a flow-rate control zone situated between the detection zone and the feedback zone, wherein each of the zones is in fluidic communication with each other with directly or indirectly by an adjacent component. Examples of absorbent articles may include, diapers, adult incontinence products, or personal or feminine hygiene products or absorbent pads for medical or hospital uses.

Alternatively, the invention describes an insert for a garment (e.g., underwear) or absorbent personal care product, the insert comprising an assay apparatus having: a lateral flow strip having a porous matrix in fluid communication with a buffer pad, wicking pad, and a flow-rate control zone situated between said buffer pad and wicking pad, said flow-rate control zone regulates an amount of time needed for development and appearance of a visual signal in a control-feedback zone of said wicking pad until a color transition in a detection zone of said buffer pad attains color stability.

Additional features and advantages of the present three-dimensional sensor or assay device and associated absorbent articles containing such a sensor will be described in the following detailed description. It is understood that the foregoing general description and the following details description and examples are merely representative of the invention, and are intended to provide an overview for understanding the invention as claimed.

### BRIEF DESCRIPTION OF FIGURES

FIG. 1 is a three-quarter schematic representation of a lateral flow assay device according to the present invention.

FIGS. 2A-C are schematic representations of different flow-rate control mechanisms which may be incorporated into the flow-rate control zone of the present assay device.

FIGS. 3A-D are enlarged schematic representations of flow-rate control mechanisms having a micro-channel pattern (similar to a brick-work pattern) to direct the fluid sample through the sensor.

FIG. 3E is an enlarged schematic representation of a combination of density or porosity gradient and micro-channel pattern.

FIG. 4 is a representation of an alternate micro-channel pattern design.

FIGS. 5A and 5B are representations of another alternate micro-channel pattern design. FIG. 5B shows an amount of fluid beginning to enter and pass through the micro-channel.

### DETAILED DESCRIPTION OF THE INVENTION

Conventional urine testing devices, such as dipsticks or test strips, operate by dipping the dipstick in a urine sample and pulling it out quickly, and then read the resultant color that can be compared with a color scale. Typically these test strips have a short reading window, typically about or less than two minutes, and do not have any user feedback mechanism. Recently, an improved hydration monitoring and test format was developed, as described in U.S. patent application Ser. No. 11/956,428, the contents of which are incorporated herein by reference. Unlike previously developed lateral flow hydration test formats, the hydration monitoring and assay device according to U.S. patent application Ser. No. 11/956,428 has a reading window with a much longer duration of at least about 2 hours, typically about 4-6 hours or greater, with stable color signal and a user feedback zone to indicate a sample volume and sample contact with the test zone. The long reading window and long term stability, of the color signal and user feedback mechanism are important features for an over-the-counter (OTC) test format, in particular, for a test in a personal care product, where constant monitoring is not practical.

### I. Lateral Flow Format

The present invention pertains to, in part, an assay apparatus that monitors specific gravity of a urine sample. The apparatus includes: a lateral flow strip having a porous matrix in fluid communication with a buffer pad, wicking pad, and a flow-rate control zone situated between the buffer pad and wicking pad. The flow-rate control zone regulates the flow rate of the sample between the buffer pad and wicking pad, therefore, it is capable of regulating the period of time that the sample is in a full contact with a detection zone of the buffer pad, and allows the assay reaction to reach a stable signal before the development and appearance of a visual signal in a control-feedback zone of the wicking pad. The detection zone can use a pH indicator that exhibits a color change responding to different pH levels. The sample observation-feedback zone has a non-diffusively immobilized pH indicator and pH adjuster. The pH indicator exhibits a color transition upon contact with the urine sample. The flow-rate control zone has a porosity gradient differential relative to the adjacent buffer pad or the wicking pad. The flow-rate control zone can be fabricated from a variety of materials, such as a nitrocellulose membrane, fiberglass pad, nylon membrane, cellulose pad, filter paper, nonwoven material, or polymeric film.

The present invention builds upon the successes of the improved lateral flow test format described in U.S. patent application Ser. No. 11/956,428, and addresses some of its shortcomings. That is, the relatively long time that the device needs to achieve a fully-developed signal state (typically ˜10 to 15 minutes). This presents a potential problem for the test integrated in personal care products. For instance, a parent may open the diaper and start to read the color signal right after a baby urinates to initiate the test without waiting for the test to reach a fully developed color signal. The present invention retains all the advantages of a lateral flow device for dehydration monitoring. The dehydration test device has a buffer pad where a buffer is loaded in a porous matrix. The buffer's pH experiences change with different ion concentrations or ion strengths or specific gravity of a sample. The buffer may consist of partially neutralized weak polymeric acid or base. Examples of weak polymeric acids or bases include poly(acrylic acid), poly(maleic acid), poly(vinylamine) and poly(4-vinylpyridine). The buffer may consist of non-polymeric weak acids such as 2-(N-morpholino)-ethanesulfonic acid and bis-(aminoethyl)-glycol ether N,N,N′,N′-tetraacetic acid. The buffer components may or may not permanently be immobilized on the porous matrix. Examples of porous matrices include cellulose pads, filter papers, non-woven materials and glass fibers pads.

The porous matrix should not interfere significantly with the association and dissociation constant of the buffer. The dehydration test device has a test pad (zone) that non-diffusively immobilizes with a pH indicator. The pH indicator desirably exhibits a color transition around neutral pH, or at a pH from about 5.5 to about 10.5. Examples of the pH indicator include bromothymol blue, thymol blue, m-cresol purple, brilliant yellow and neutral red. The matrix is preferred to be porous and urine (aqueous) friendly to allow rapid penetration of urine.

The hydration/dehydration test device has a porous and hydrophilic wicking pad. The wicking pad is preferred to have a relatively high or significant absorbent capacity of holding fluids, such as water or urine. The hydration/dehydration test device has an observation feedback pad (zone) that can change color upon contact with urine regardless of the pH and/or specific gravity of the urine sample.

The sample observation-feedback pad can have a non-diffusively immobilized pH indicator and a pH adjuster on a porous and water/urine friendly matrix. The pH indicator can exhibit a color transition at a pH either less than about 5.5 or greater than about 10.5. Examples of the pH indicator include bromophenol blue, bromochlorophenol blue, phloxine B, Bromocresol green and Congo red. Examples of the pH adjuster include citric acid, oxalic acid and tartaric acid. The matrix is preferred to be porous and urine friendly to allow rapid penetration of urine.

The hydration/dehydration test device has a flow-rate control zone that can regulate the flow rate of a sample from the buffer pad to the wicking pad. Unlike previous generation of lateral flow dehydration tests that provide feedback only for the amount of sample and sample contact with the test zone, the current invention provides a device which also prevents the false reading because of lack of enough signal developing time for the detection zone. According to the present invention, several methods exist to regulate the flow rate. One example is a piece of porous membrane to be bridged between the buffer pad and wicking pad. The membrane's pore size, width and length of the zone, geometry of the zone, wettability of the pore surface and their combination can be used to tailor the liquid flow rate from the buffer pad and wicking pad. Examples of the porous membranes include nylon membranes, cellulose-based papers and tissues, fiber-based nonwoven materials. The hydration/dehydration test device may also have a supporting substrate that helps secure those components together to make an integrated device.

### II. Error-Reduction & Lateral Flow Control

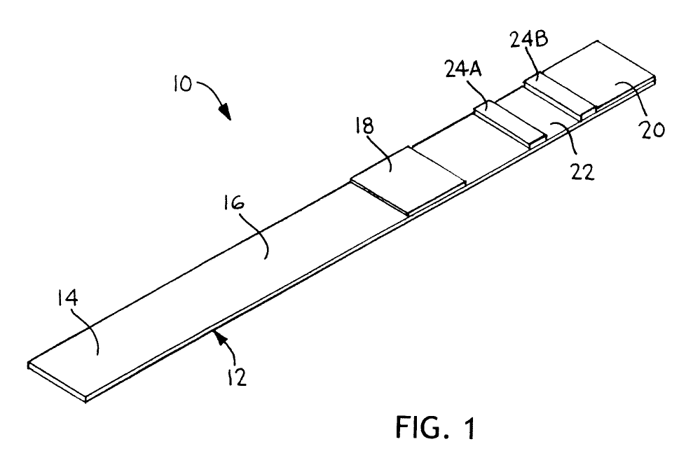

A schematic illustration of an example of a lateral flow assay device according to the present invention is shown in accompanying FIG. 1. The device 10 has a supporting substrate 12 upon which are located a sample deposition zone 14, a buffer zone or pad 16, a detection zone 18, and a sample reading or observation-feedback zone 20. Situated between the detection zone 18 and sample reading-observation feedback zone 20 is a flow-rate control zone 22, each respectively with a porous pad or membrane. According to an embodiment, overlapping areas 24a, 24b are located before and after the flow-rate control zone in reference to the direction of general fluid flow, from a first end A near the sample deposition zone to a second end B near the feedback zone zone.

In particular, the buffer pad 16 is laminated on one side of the supporting substrate 12 and a wicking pad is laminated on the other side of the substrate. Situated in between the two is a porous membrane with an overlap 24a with the buffer pad at one end and an overlap 24b with the other end. The signal indicator in the feedback pad is situated in the example on the top of the wicking pad. A sample test pad is situated over a portion of the buffer pad to form a detection zone. These components are laminated together, for instance with a liquid-insoluble adhesive or tape. The buffer pad, the test pad, the flow-rate control membrane, the wicking and feedback pad are in fluid communication, either directly or in directly through other components. A portion of the buffer pad can also act as a sample zone where sample is introduced. As an alternative, a separate sample pad can be laminated with the buffer pad with fluid communication between them. The whole device is enclosed in a sealed casing to prevent sample evaporation except in the sample deposition zone. The end of the wicking pad is preferred not to be completely sealed, but with a small hole or aperture, so that the end is in contact with air. In an alternate embodiment, the dehydration test device can be integrated as a part of a personal care product, such as a diaper, children's training-pants, adult incontinence or health care product, or used as an exchangeable insert in such products.

In traditional lateral flow devices, the flow rate is constant from one end of the device to the other. Typically in many lateral flow-based assay, for the tests to perform properly and to their full capabilities, it may require about 5-10 minutes to reach reaction equilibrium and to stabilize their signal. The consumer/user may not be aware of this time delay, and so may perceive an erroneous result. An object of the present invention is to minimize errors that arise from premature reading. In the present invention, one can modify and regulate the flow rate of flow of a urine sample through the lateral flow device to serve as a timing mechanism. A function of the flow-rate control zone is to regulate the progress of a liquid sample to reach the feedback pad for feedback signal development by about 1 or 2 minutes to about 20 or 30 minutes, and potentially by about 1 or 2 hours up to about 8 or 10 hours, depending on the desired application. This can help the consumer determine what time is a valid time to read the signal. Further, this can help the consumer compare a stabilized signal in the detection zone to one that manifests in the sample observation zone. In other words, the flow-rate control cone permits the detection zone to perform to its optimal stability.

The flow-rate control zone can be configured as an interchangeable, separate assembly or component that either the manufacturer can tailor for particular applications. According to an embodiment, the flow-rate control zone has a discrete membrane, separate from the material substrate of the wicking pad, but in fluidic communication with the sample flow. Alternatively, the flow-rate control zone can integrated as a permanent inset part of the lateral flow assay substrate. To adjust the speed of liquid permeation, one may employ a difference in substrate porosity. The materials in the flow-control zone can be calibrated to provide a predetermined rate so as to enable one to know beforehand or have a preset timer to know when to read a signal. Such as wait until at least a second control line is developed for signal validation in the dehydration sensing lateral flow substrate. Depending on the nature of the substrate material, one can tailor the reactivity of the materials with sample fluids or mechanisms of the reactions at the detection zone.

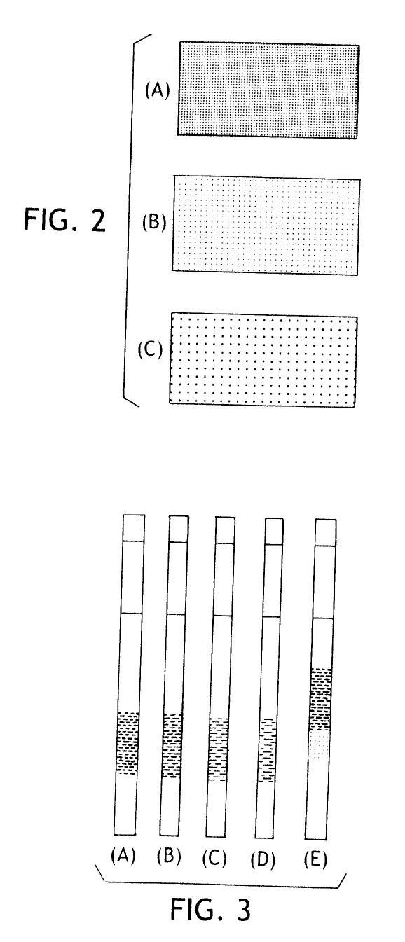

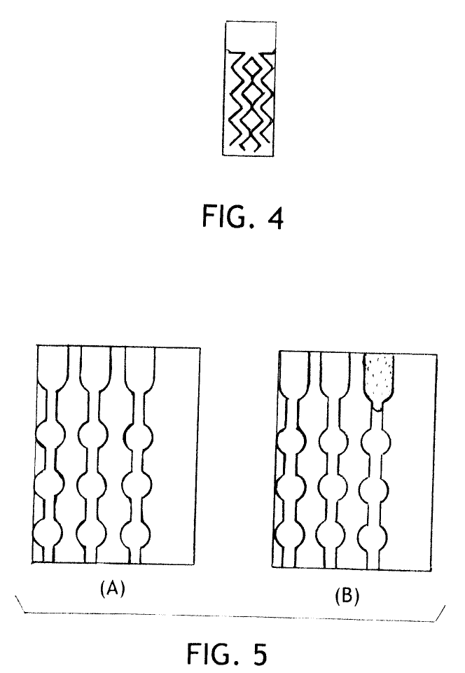

A number of different devices or mechanisms may be employed in the flow-rate control zone 22. The devices can be arrayed as surface features of the flow-rate control zone or arranged in laminated layers to provide some thickness and to either assist or hinder the progress of capillary transport of a sample liquid. The devices can either increase or decrease the time that it may take for a sample volume to traverse from the buffer and wicking pads through the flow-rate control zone 22 to the sample observation or control zone 20. For example, such as represented in FIGS. 2-4, flow-rate control devices can be take the form of a density gradient, filter gradient of varying degrees of porosity, such designs of micro-channel patterns either in parallel, orthogonal, on a diagonal, or in combination to a primary direction of lateral flow, and a combination thereof. These devices can be incorporated either as a number of parallel, orthogonal, or diagonally oriented micro-channel patterns along the surface of the flow-control zone or layered in the body of the flow-rate control zone of each assay unit to help regulate the flow rate of the liquid from one side of the lateral flow assay to the other, along the primary flow direction. The micro-channels can have a cross-sectional dimension from about 0.01 microns to about 50 or 60 microns. Typically, the cross-sectional dimension may range from about 0.5 micron to about 35 or 40 microns, or from about 1-3 or 5 microns to about 20-22 or 25 microns. The micro-channels may encourage mixing of fluids. Parallel oriented flow-rate control devices can be placed to follow the general direction of liquid flow, such that the liquid can travel largely along one plane from the detection zone to sample observation control zone, such as depicted in FIGS. 3-5. In contrast, orthogonally oriented flow-rate control devices can be situated largely perpendicular to the liquid flow direction such that the liquid passes through the plane of each horizontal layer, such as depicted in FIG. 1. Laminations can include various types of tapes and polymer films. The present device can include a large number or combination of layers of laminated substrates, each with a particular physical or chemical property, as long as the layers are in fluid contact with each other. The lamination can involve forming a number of flow-rate control devices as features in a substrate surface and joining a plurality of substrate layers together in laminate structure.

FIGS. 2A-C are schematic representations of different flow-rate control mechanisms which may be incorporated into the flow-rate control zone of the present assay device. In particular, the figures represent configurations with relative density or porosity of the filter medium in the flow-rate control zone. FIG. 2A represents a relatively high density, about 2,100 or 2300 to about 2,500 or 2.700 per square inch; FIG. 2B represents a medium density, about 1,100 or 1,200 to about 1,900 or 2,000 per square inch; and FIG. 2C represents a relatively low density of about 1,000 or 1,100 per square inch or less. Each dot represents either a concave pocket or convex nodule.

The physical dimensions of the flow-rate control and associated overlapping regions on the lateral flow assay device can vary depending on the desired application and absorbent needs. Physical dimensions along x-y directions, defining a plane, can be any size that is area large enough to satisfy the needs of a specific use. Physical dimensions along the thickness or z-direction should be sufficient to accommodate the volume of liquid in a sample; which typically is a faction or the whole thickness of a substrate body of a testing article. For instance, possible practical dimensions along the x-y direction can range from as short as a few millimeters (e.g., ˜1-7 mm) or up to a few centimeters (e.g., about 1-4-5-7 or 10 cm). Typically, the length dimensions are between about 2 or 3 cm for each side as desired. The overall flow-rate control zone can have an area of about 1-4 cm² up to about 100 cm² (e.g., about 2-3-5-8-10-12-16-20-25 cm²) as desired. The thickness of the flow-rate control zone may range from about 0.01 or 0.04 cm up to about 1.0 or 2.0 cm thick (e.g., about 0.1-0.25-0.50-0.8-1.5 cm).

The flow-rate control devices can be generated in or on the porous substrate by means of a variety of methods or processes, such as die cuts, laser etching, chemical etching, or printing reagents on the substrate surface. The particular method of creating the flow-rate control devices may depend on the chemical or physical nature of the substrate material (i.e., porosity, hardness, chemical reactivity/composition) and the desired geometries, shapes, patterns, ablation depths.

According to certain embodiments, the flow-rate control mechanisms of the present invention involve physically altering the lateral flow membrane so that particular fluid flow rates and flow patterns. One of the major advantages of the present flow-rate control mechanism is that it does not need to introduce special or additional materials into current lateral flow assay products. All of the physical alterations are made to the existing testing membrane, such as mentioned above, appropriate nitrocellulose membranes, glass fiber pads, cellulose pads, non-woven of thermoplastic polymers, or filter papers. Another advantage of the present invention is that it could be implemented into existing manufacturing processes without requiring large recapitalization of equipment. A third advantage of this invention is that it can be seamlessly included into the test without negatively affecting the performance or the accuracy of the test. It can be applied to numerous testing formats, including immunoassays, chemical assays, small molecule detection, nucleic acid testing, and others.

One embodiment of this invention involves using a laser to remove specific sections of one or more of the lateral flow membranes. To maintain test integrity, the laser intensity can be tuned so that only the membrane is removed and not the supporting backing card. The membrane can be removed in sections or patterns of laser cuts can be created. Examples of patterns include, but are not limited to dots, dashes, circles, triangles, other geometric shapes. Additionally, the laser can be dynamically tuned such that a three dimensional relief of a picture or complex pattern is achieved. The density and cross-sectional depth of these patterns can effectively control the rate of the flow of the fluid sample. In addition, the pattern and shape of the laser cuts can control the direction of fluid flow on the test strip.

FIG. 2 shows examples of patterns created on a laminated nitrocellulose test strip with a laser. Other methods of physically altering the membrane through mechanical means include punch dyes, vinyl cutters and others. Another embodiment of this invention utilizes printing techniques to apply hydrophobic materials to the lateral flow membrane material. The hydrophobic materials can be applied in sections or patterns much like the laser cut embodiment. Similarly, the pattern, the density, and the shape can control and alter flow rate and flow direction of the fluid. Additionally, the penetration depth of the hydrophobic ink into the membrane can change the rate of fluid flow. Examples of the hydrophobic materials include but are not limited to commercial Sharpie® ink and hydrophobic polymers (e.g., polystyrene and polyvinyl chloride). An example of a permanent ink (e.g., Sharpie™ Ink) on nitrocellulose is depicted in FIG. 3. Yet another embodiment includes removing sections of the lateral flow membrane through chemical etching. An example of this methodology was previously described in two U.S. Patent Publications US 2006/0246597 A1, and US 2006/0246600 A1, the content of which are incorporated herein by reference, describing flow control and metering techniques, respectively.

In particular, one or more recessed regions are formed in the substrate by applying a solvent treatment. The solvent treatment is selected based on its particular dissolving capacity for the material used to form the membrane. For example, an alcohol-based solvent, such as methanol, may be used for nitrocellulose membranes. Upon contact with the solvent treatment, a recessed region is formed that may serve a variety of different functions relating to flow-rate control. The solvent treatment may be applied to the membrane using any of a variety of well-known application techniques. Suitable application techniques include, for example, standard lithography and photo resist technology, spraying, printing (e.g., inkjet, pad, etc.), pipetting, air brushing, metering with a dispensing pump, and so forth. In one particular embodiment, for example, the solvent treatment is applied using a dispensing and optional drying process commonly employed to form detection lines on lateral flow strips. Such a system could involve placing a sheet of the porous membrane on a dispensing machine and threading it through a rewind spindle. This may be accomplished using either a batch or continuous process. The dispensing machine delivers a precise volume of the solvent treatment in a straight line as the membrane passes beneath. The sheet then passes through a drier and is wound back on a spool for further processing. For instance, a lab-scale dispensing pump system for batch processes is available from Kinematic Automation, Inc. of Twain Harte, Calif. under the name “Matrix® 1600.”

The solvent treatment may also be applied in any amount effective to form a recessed region having the desired size and shape. The ultimate amount employed may depend on a variety of factors, including the dissolving capacity of the solvent for the membrane material, the speed of application, etc. Regardless of the manner in which it is formed, the recessed region generally acts as a flow-rate control mechanism for the lateral flow device. For example, the recessed region may block the flow of the fluid through the membrane until such time that the assay is initiated, such as by placing the membrane in fluid communication with another membrane. Alternatively, the recessed region is simply used to slow down or otherwise control the flow of fluid through the membrane. For example, a plurality of discrete recessed regions (e.g., dots) may be formed to reduce the continuity of the membrane structure. Thus, a fluid flowing through the membrane structure is forced to follow a tortuous pathway, which increases the amount of time for the fluid to reach the detection zone. Such an increased flow time may provide a variety of benefits, such as to promote uniform mixing and ensure that an) analyte within a test sample has sufficient time to react with the desired reagents. For example, the time for the test sample to reach the detection zone may be at least about 1 minute, in some embodiments at least about 2 minutes, in some embodiments from about 3 or 5 minutes to about 8 or 10 minutes, and in some embodiments, from about 10 or 12 minutes to about 25-30 minutes.

Particular uses for the present invention, it is envisioned, may include any lateral flow assays in which timing is critical, such as ensuring a minimum time has elapsed before reading and interpreting the results. One such example is a dehydration test designed for inclusion in a personal care garment, such as a diaper, where precise monitoring of the test is not practical. For a dehydration indicator developed internally, the detection zone requires 5-10 minutes to stabilize and reach equilibrium after coming n contact with the urine sample. If the test is read prior to equilibrium, inaccurate results may be given. Thus, it would be useful to include one of these flow-rate control zones in between the detection zone and the sample observation-control zone such that the sample fluid would not react with the sample observation-control zone until 10 minutes after reaching the detection zone. In such an embodiment, the user would be assured that the test is ready to read once the observation-control zone color has formed. The flow-rate control zones could also be used in between detection zones of a multi-analyte test in which the signal from the zones forms at different rates. In such situation, it would be advantageous that most or all of the signals develop at the same time, so as not to confuse the user. Otherwise, the user may assume the test is complete once one signal is formed and therefore miss the other signals that develop later.

Although the present inventive concept is described in terms of solving issues associated with reading lime for a dehydration test to be incorporated into an absorbent article (e.g., diaper, or adult incontinence product), the concept has potential for broader applications. Additionally, applications for this technology are envisioned to reach beyond lateral flow technology and diagnostic applications. Potentially, this technology could be used in any type of situation where filtering is required or where fluid flow rates need to be controlled.

In another aspect, the invention also relates to a method for testing specific gravity of a urine sample, the method comprises: introducing a urine sample to a sample zone, passing said urine through a buffer pad in a detection zone, causing a color change in a pH indicator in said detection zone, passing the urine through a flow-rate control zone to regulate the appearance of a visual signal in an observation-feedback zone of the wicking pad (for a predetermined interval), until a color transition in the detection zone attains color stability.

The testing is normally performed according to the following: A urine sample is introduced into the sample zone and flows through the buffer zone through capillary action. The ions in the urine cause the change of the buffer's pH in the buffer pad. Some of the samples flows into the detection zone where the pH indicator will show different colors depending upon the pH of the buffer, which is determined by the ion concentration of the urine sample. It is the color of the detection zone that correlates with the urine ion strength, or specific gravity of the urine, which reflects a person's hydration status. It was found that the color signals in the detection zone normally take some time (normally 10 to 30 minutes depending upon the device dimension and configuration) to be fully developed. Some of the sample further flows to the flow-rate control zone, then to the wicking zone, and then to the sample observation-control zone to finally trigger a color change in the reading zone. The time it takes for the sample to fully reach the observation-feedback zone to develop the feedback signal call be easily regulated through many parameters of the flow-rate control zone, including the selection of the material, width and length of the zone and pore size. The color change in the feedback pad can be used to provide not only assurance that the test is properly done, but also to ensure a minimal time that the sample has contacted with the detection zone before reading the signal. For instance, the test is not valid if the feedback pad has not experience a color change, indicating that one either did not have sufficient amount of sample introduced or had not allowed sufficient time for the signal to develop in the detection zone.

### III. Example

### 1. Preparation of Components:

A 1 cm×30 cm piece of cellulose pad from Millipore Co. is soaked with 5 ml of polyacrylic sodium salt that is titrated to pH of 8.1 with 1N HCl. The pad is air-dried overnight to make a buffer pad. A 10 cm×10 cm piece of Biodyne Plus Nylon membrane from Pall Co. is soaked in a 30 ml of bromothymol blue aqueous solution (0.1 mg/ml) for 10 minutes and air-dried overnight to make a test pad. A 10 cm×10 cm piece of Biodyne Plus membrane is soaked with an aqueous solution containing bromocresol green (0.2 mg/ml) and citric acid (2 mg/ml) for 10 minutes and air-dried overnight to make a control test pad.

2. Assemble the Device with a 3 mm Wide Flow-Rate Control Zone:

On an 8 cm×30 cm supporting plastic card was laminated with a 5 mm wide strip of Biodyne B membrane. 2 cm from the edge of the card to make a flow-rate control zone. A 6 mm wide strip of a cellulose wicking pad was laminated on the card with a 1 mm overlap with the Biodyne B membrane on one side. A 2 cm wide buffer pad was laminated on the other side of the Biodyne B membrane with 1 mm overlap with the Biodyne B membrane to make a buffer zone. A 2 cm wide cellulose sample pad was laminated with 5 mm overlap with the buffer pad to make a sample zone. A 5 mm wide strip of the test pad was laid on the top of the buffer pad, 2.2 mm from the edge and secured by a Scotch tape, to make a test zone. A 5 mm wide strip of the sample control pad is laid on the top of the wicking pad and secured by a tape. The card was cut into 5 mm wide devices. The devices were sealed by tape except the sample zone. The particular embodiment creates a multi-layered structure with sections of the lateral flow device adjacent to the flow-rate control zone overlapping the flow-rate control substrate material.

3. Assemble the Device with an 8 mm Wide Flow-Rate Control Zone:

All the steps are the same as above except using a 10 mm wide Biodyne B membrane to replace the 5 mm wide Biodyne B membrane to make a flow-rate control zone.

4. Test the Devices with 3 mm Wide Flow-Rate Control Zone:

To each of six wells in a microtiter plate was added 200 μl of synthetic urine with a specific gravity of 1.002, 1.008, 1.014, 1.020, 1.025 and 1.035, respectively. A dehydration test device was inserted into each sample well. About two minutes later, the color in the detection zone started to develop. Five minutes later, the sample observation-control zone shows no color change. About ten minutes later, a small portion of the observation-control zone started to show color change from yellow to blue. At this time, the color signal in the detection zone reaches stability. About 15 minutes later, the whole control zone became blue.

5. Test the Devices with 8 mm Wide Flow-Rate Control Zone:

The results were similar except that the sample observation-control zone started to show color change at about 15 minutes after sample application and at about 20 minutes the whole sample observation-control zone showed color change.

In summation, the present inventive concept describes a fluidic testing device, an absorbent article incorporating the test device and a process for controlling flow rate and flow path of a fluid sample. The testing format and process that involves: providing porous substrate material which transports a fluid sample along a pathway from a point of deposition to at least one detection zone by means of capillary action; modifying a section of said pathway to a) create a physical pattern of channels either on or in said porous substrate material, b) provide varying density and cross-sectional depth of said porous substrate, or c) a combination of a) and b). The substrate material includes at least a portion having a porous membrane with a flow-rate and flow-path control mechanism, either as part of the substrate or as a separate laminate layer in fluid communication with adjacent components.

The present invention has been described both generally and in detail by way of examples and the figures. Persons skilled in the art, however, can appreciate that the invention is not limited necessarily to the embodiments specifically disclosed, but that substitutions, modifications, and variations may be made to the present invention and its uses without departing from the spirit and scope of the invention. Therefore, changes should be construed as included herein unless the modifications otherwise depart from the scope of the present invention as defined in the following claims.

1. A testing device for monitoring hydration or dehydration, the device comprising:
a first substrate with a porous matrix adapted for conducting lateral flow, said substrate having a sample contact zone, a detection zone, feedback zone, and a flow-rate control zone situated down stream from said detection zone, between said detection zone and said feedback zone, spatially separating said detection and feedback zones, wherein each of said zones is in fluidic communication with each other, either directly or indirectly by an adjacent component, such that a sample can travel from said detection zone to said flow-rate control zone before developing a visual signal in said feedback zone.

2. The testing device according to claim 1, wherein said detection zone is part of a buffer pad situated between said sample contact zone and said flow-rate control zone.

3. The testing device according to claim 1, wherein said observation-feedback zone is part of a wicking pad.

4. The testing device according to claim 3, wherein said wicking pad further includes an observation-control zone that changes color upon contact with urine regardless of specific gravity of the urine.

5. The testing device according to claim 1, wherein said detection zone has a pH indicator that exhibits a color transition at a pH from about 5.5 to about 10.5.

6. The testing device according to claim 4, wherein said observation-control zone has a non-diffusively immobilized pH indicator and pH adjuster, said pH indicator exhibits a color transition at a pH of either less than 5.5 or greater than 10.5.

7. The testing device according to claim 1, wherein said flow-rate control zone is at least part of a second substrate separate from said first substrate.

8. The testing device according to claim 1, wherein said flow-rate control zone regulates the flow rate from said buffer pad to said wicking pad.

9. The testing device according to claim 8, wherein said flow-rate control zone is a porous membrane that bridges a gap between said buffer pad and said wicking pad.

10. The testing device according to claim 1, wherein said flow-rate control zone has a number of flow-rate control mechanisms that regulate an amount of time needed for development and appearance of a visual signal in said observation-feedback zone until said color transition in said detection zone reaches color stability.

11. The testing device according to claim 1, wherein said flow-rate control zone mechanisms control the flow rate by means of manipulating porosity, density, or ion affinity gradient in a matrix forming at least part of said flow-rate control zone.

12. The testing device according to claim 1, wherein said flow-rate control zone is made from a nitrocellulose membrane, fiberglass pad, nylon membrane, cellulose pad, filter paper, nonwoven material, or polymeric film.

13. The testing device according to claim 1, wherein a supporting member secures each of the zones together in an integrate device.

14. A method of monitoring hydration, the method comprises: providing a later flow strip with a porous matrix in fluid communication with a buffer pad, wicking pad, and a flow-rate control zone situated between said buffer pad and wicking pad; introducing a test sample to a sample zone on said buffer pad, allowing said sample to seep through a detection zone to said flow-rate control zone before developing a visual signal in an observation-feedback zone; controlling the flow rate by means of manipulating porosity, density, or ion affinity gradient in a matrix forming at least part of said flow-rate control zone.

15. A method for testing ion strength of a urine sample, the method comprises: introducing a urine sample to a sample zone, passing said urine through a buffer pad in a detection zone, causing a color change in a pH indicator in said detection zone, passing said urine through a flow-rate control zone to regulate the time needed for appearance of a visual signal in a control-feedback zone of said wicking pad until a color transition in said detection zone attains color stability.

16. An absorbent article comprising a test device for monitoring hydration or dehydration, the device comprising: a first substrate with a porous matrix adapted for conducting lateral flow, said substrate having a sample contact zone and a detection zone as part of a buffer pad, feedback zone as part of a wicking pad, and a flow-rate control zone situated downstream of said detection zone, between said detection zone and said feedback zone, wherein each of said zones is in fluidic communication with each other either directly or indirectly by an adjacent component such that said flow-rate control zone regulates development of a visual signal in said feedback zone.

17. The absorbent article according to claim 16, wherein said detection zone is situated between said sample contact zone and said flow-rate control zone.

18. (canceled)

19. The absorbent article according to claim 16, wherein said wicking pad further includes an observation-control zone that changes color upon contact with urine regardless of specific gravity of the urine.

20. The absorbent article according to claim 16, wherein said flow-rate control zone has a number of flow-rate control mechanisms that regulate an amount of time needed for development and appearance of a visual signal in said observation-feedback zone until said color transition in said detection zone reaches color stability.

21. The absorbent article according to claim 20, wherein said flow-rate control mechanisms control the flow rate by means of manipulating porosity, density, or ion affinity gradient in a matrix forming at least part of said flow-rate control zone.

22. The absorbent article according to claim 19, wherein said article is a personal care product selected from: diapers, adult incontinence products, feminine hygiene products, or absorbent pads.

23. An insert for a garment or absorbent personal care product, the insert comprising an assay apparatus having: a lateral flow strip having a porous matrix in fluid communication with a buffer pad, wicking pad, and a flow-rate control zone situated between said buffer pad and wicking pad, downstream of a detection zone, said flow-rate control zone regulates an amount of time needed for development and appearance of a visual signal in a control-feedback zone of said wicking pad until a color transition in a detection zone of said buffer pad attains color stability.

24. An absorbent article that includes an insert comprising an assay apparatus having:
a lateral flow strip having a porous matrix in fluid communication with a buffer pad, wicking pad, and a flow-rate control zone situated between said buffer pad and wicking pad, downstream of a detection zone, said flow-rate control zone regulates an amount of time needed for development and appearance of a visual signal in a control-feedback zone of said wicking pad until a color transition in a detection zone of said buffer pad attains color stability.

25. The absorbent article according to claim 24, wherein said flow-rate control has a combination of layers of laminated substrates, each with a particular physical or chemical property.
